(19) United States
(12) Patent Application Publication
(10) Pub. No.: US 2024/0189217 A1
(43) Pub. Date: Jun. 13, 2024

(54) SUNSCREEN COMPOSITION INCLUDING A FLUORESCENT AGENT EXCITABLE BY AN ULTRAVIOLET LAM

(71) Applicants: Zachary Shulman, San Diego, CA (US); Steve Shulman, San Diego, CA (US)

(72) Inventors: Zachary Shulman, San Diego, CA (US); Steve Shulman, San Diego, CA (US)

(21) Appl. No.: 18/077,379

(22) Filed: Dec. 8, 2022

(51) Int. Cl. A61K 8/96; A61K 8/19; A61Q 17/04

(52) U.S. Cl. CPC A61K 8/965; A61K 8/19; A61Q 17/04; A61K 2800/434; A61K 2800/81; A61K 2800/87

(57) ABSTRACT

Compositions, devices, and methods related to determining adequacy of the application of a sunscreen composition on human skin are disclosed. A disclosed sunscreen composition includes a sunscreen agent and a fluorescent agent, which, when excited by an ultraviolet light having a compatible wavelength, fluoresces in visible light. A method for determining whether the sunscreen composition has adequately applied on human skin is disclosed. A disclosed device generates an ultraviolet light having a compatible wavelength and is portable.

### BACKGROUND

A wide range of electromagnetic radiation from the sun reaches the earth. One type of radiation called ultraviolet (UV) radiation or rays (invisible to the human eye) is most damaging to human skin. On the sun's electromagnetic spectrum, the ultraviolet region is between visible light and X-rays, with wavelength from 10 and 400 nm. The ultraviolet region comprises three sections: UVA (320-400 nm), UVB (290-320 nm), and 100-290 nm). UVC rays are the most dangerous of the ultraviolet rays but are absorbed by the ozone layer and other gases in the earth's atmosphere. Unlike UVC rays, UVA and UVB rays reach the earth's surface. UVB rays act directly on biological molecules on human skin, causing skin cancer, skin ageing, and delayed sunburn that arises 12-24 hours after exposure. UVA rays penetrates deeper into the skin and damages blood vessels and DNA. UVA rays also play a role in skin cancer formation.

A wide-ranging sunscreen compositions, in forms of lotion, gel, cream, and spray, are available in the market to protect against the harmful damages caused by the sun's ultraviolet radiation. These compositions typically incorporate one or more sunscreen agents designed to block out UVA and UVB rays. However, the efficacy of these compositions depends on how well they are applied to skin. Typically, a small amount of such a composition is rubbed, by hand, over areas of the skin that are exposed to the sun to create a film or layer of protection. Proper application of sunscreen composition requires an even and complete coverage of all skin areas which are exposed to the sun. However, it is often the case that some portions of the exposed skin areas are missed during the rubbing process and therefore, are not protected by the composition. Further, typically, sunscreen compositions are colorless or otherwise blend into the color of the skin, when applied, making it difficult to detect the missed spots by the naked eye. It is thus difficult for a user of these typical sunscreen compositions to assure even and complete coverage on the skin.

It is, therefore, an object of the invention to provide a sunscreen composition which increases the likelihood of even and complete coverage when applied to the skin. Another object of the invention is to provide a device and method to detect, by the naked eye, whether the sunscreen composition has been properly applied to the skin and whether there are missed spots. Still another object of the invention is to ensure that the detection device is portable in that it may be used in at home and in the field (e.g., at a beach) as opposed to in a laboratory setting where expensive and bulky equipment, such as a spectrometer, is used. These and other objects of the invention will become apparent in the description which follows.

### SUMMARY

Compositions, devices, and methods related to determining the adequacy of the application of those compositions on human skin are disclosed. The compositions disclosed herein comprise a sunscreen agent that minimizes the harmful effects of the sun's ultraviolet light and a fluorescent agent, which, when excited, i.e., irradiated, by an ultraviolet light having a compatible wavelength, fluoresces in visible light that is observable by the naked eye. Visible light is usually defined as having wavelengths in the range from around 400 nm to around 780 nm. For example, within this range, typically a visible light can be of the following colors: violet (400-420 nm), indigo (420-440 nm), blue (440-490 nm), green (490-570 nm), yellow (570-585 nm), orange (585-620 nm), and red (620-780 nm). The devices disclosed herein generate an ultraviolet light having a compatible wavelength and are portable in that can be used at home and in the field (as opposed in a laboratory setting). The methods for determining whether the compositions have been adequately applied on human skin are disclosed.

In accordance with specific embodiments of the invention, a sunscreen composition for application to human skin is provided. The composition comprises: a sunscreen agent selected from the group consisting of UVA sunscreen agent, UVB sunscreen agent, UVA-UVB sunscreen agent, and mixture thereof; and a fluorescent agent in an effective amount; wherein the fluorescent agent emits a visible light when irradiated with an ultraviolet light having a compatible wavelength.

In accordance with specific embodiments of the invention, a method for determining adequacy of an application on skin of a sunscreen composition is provided. The sunscreen composition comprises: (i) a sunscreen agent selected from the group consisting of UVA sunscreen agent, UVB sunscreen agent, UVA-UVB sunscreen agent, and mixture thereof, and (ii) a fluorescent agent in an effective amount, wherein the fluorescent agent emits a visible light of a predetermined color, when irradiated with an ultraviolet light having a compatible wavelength. The method comprises: manually applying a recommended amount of the sunscreen composition to an area of the skin; imparting, using a UV lamp, the ultraviolet light on the area of the skin; and visually inspecting, during the imparting the ultraviolet light, the area of the skin to determine if the predetermined color does not appear in any subregion of the area of the skin.

In accordance with specific embodiments of the invention, a portable device is provided. The portable device comprises: a container containing a sunscreen composition, the sunscreen composition comprising (i) a sunscreen agent selected from the group consisting of UVA sunscreen agent, UVB sunscreen agent, UVA-UVB sunscreen agent, and mixture thereof, and (ii) a fluorescent agent; and a UV lamp attached to the container; and wherein the UV lamp, when powered on, emits an ultraviolet light having a compatible wavelength; wherein the sunscreen composition emits a visible light when irradiated with the ultraviolet light emitted by the UV lamp.

### BRIEF DESCRIPTION OF THE DRAWINGS

FIG. 1 illustrates an example of container for packaging and containing sunscreen composition and a UV lamp integrated in the container, in accordance with specific embodiments disclosed herein.

FIG. 2 illustrates an example cross-sectional view of the container in FIG. 1.

FIG. 3 illustrates an example of a container for packaging and containing sunscreen composition and a detachable UV lamp, in accordance with specific embodiments disclosed herein.

FIG. 4 illustrates a flowchart for a method for determining adequacy of an application of a sunscreen composition on skin, in accordance with specific embodiments disclosed herein.

### DETAILED DESCRIPTION

Reference will now be made in detail to implementations and embodiments of various aspects and variations of compositions, devices, and methods described herein. Although several exemplary variations of the compositions, devices, and methods are described herein, other variations of the compositions, devices, and methods may include aspects of the compositions, devices, and methods described herein combined in any suitable manner having combinations of all or some of the aspects described.

The compositions, devices, and methods disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

In accordance with various embodiments of the invention, the sunscreen composition can be lotion, gel, cream, or spray. The sunscreen composition, in accordance with various embodiments of the invention, comprises a sunscreen agent that is suitable for application to skin and can protect against or minimize the above-described harmful effects of UVA rays or UVB rays, or both. Two kinds of sunscreen agents are used in the sunscreen compositions currently available in the market for minimization of these harmful effects: organic (chemical) agents, e.g., octyl methoxycinnamate, benzophenone-3 or octocrylene, which absorb light in the UV range, and inorganic (physical) agents, zinc oxide and titanium dioxide, which scatter and reflect UV rays. Currently available sunscreen compositions usually comprise more than one of these agents: organic, inorganic, or a mixture of both types, which gives broad-spectrum of protection.

In specific embodiments of the invention, the sunscreen composition comprises a UVA sunscreen agent that protects against or minimizes the harmful effects of UVA rays. Non-limiting examples of UVA sunscreen agents include: Benzophenone-1, Benzophenone-2, Benzophenone-3, Benzophenone-4, Benzophenone-6, Benzophenone-8, Butyl methyl dibenzoyl methane, Disodium phenyl dibenzimidazole tetrasulfonate, Menthyl anthranilate, 4-isopropyl dibenzoyl methane, and Terephthalylidene dicamphor sulfonic acid.

In specific embodiments of the invention, the sunscreen composition is a UVB sunscreen agent that protects against or minimizes the harmful effects of UVB rays. Non-limiting examples of UVB sunscreen agents include: DEA methoxyinnamate, Ethyl dihydroxypropl PABA, Glycerol PABA, Homosalate, Octocrylene, Octyl dimethyl PABA, OCtyl methoxycinnamate, Octyl salicylate, PABA, 2-Phenyl-benzimidazole-5-Sulphonic acid, TEA Salicylate, 3-(4-methylbenzlidene)camphor or 3-(4-methylbenzylidene)boran-2-one, and Etocrylene.

In specific embodiments of the invention, the sunscreen composition is a UVA-UVB sunscreen agent that protects against or minimizes the harmful effects of both UVA rays and UVB rays. Non-limiting examples of UVA-UVB sunscreen agents include: Benzophenone-3, Benzophenone-4, Benzophenone-8, 3-Benzylidene camphor, Bis-ethylhexyloxyphenol methoxyphenyl triazine, Cinoxate, Drometrizole trisiloxane, Methylene bis-benzotriazolyl, Tetramethylbutylphenol, Octocrylene, and Phenylbenzimidazole sulfonic acid.

In specific embodiments of the invention, the sunscreen composition comprises one of a UVA sunscreen agent, a UV-B sunscreen agent, a UVA-UVB sunscreen agent, or a mixture two or more of a UVA sunscreen agent, a UV-B sunscreen agent, and a UVA-UVB sunscreen agent. The total amount of sunscreen agent(s) included in the sunscreen composition can vary depending upon the specific sunscreen agent(s) is(are) used. In specific embodiments of the invention, the sunscreen agent is included in the composition at about 1 percentage by weight or weight percent (“wt. %”) to about 60 wt. % of the total composition. In some embodiments of the invention, the sunscreen agent is included in the composition at about 5 wt. % to about 20 wt. % of the total composition. In some embodiments of the invention, the sunscreen agent is included in the composition at about 2 wt. % to about 15 wt. % of the total composition.

In various embodiments of the invention, the sunscreen composition comprises a fluorescent agent in an effective amount. The term “effective amount” as used herein denotes an amount of any fluorescent agent that will confer an observable fluorescence in the presence of an ultraviolet light having a compatible wavelength and less than 150 lumens when it has been spread onto human skin. An effective amount can be from about 0.01 wt. % to about 30 wt. % of the total sunscreen composition, more preferably, however, from about 0.01 wt. % up to about 10 wt. %, most preferably about 1 wt. % to about 8 wt. %, with about 2-5 wt. % being the most commonly employed amount.

The term “compatible wavelength” as used herein denotes a peak wavelength of an ultraviolet light generated by an ultraviolet lamp (throughout the specification, occasionally referred to as an “UV lamp”) at which wavelength a fluorescent agent is excited and fluoresces, i.e., emits fluorescent light, in the visible light spectrum. Such emitted fluorescence can be observed by naked eyes. In various embodiments of the invention, a compatible wavelength can be from around 280 nm to around 400 nm (comprising long-wave and mid-wave ultraviolet lights), more preferably from around 320 nm to around 400 nm (comprising long-wave ultraviolet lights). This choice of compatible wavelengths, and corresponding fluorescent agents, is beneficial in that an UV lamp that generates an ultraviolet light having a compatible wavelength can be portable such that it can be used in the field (as opposed to in a laboratory setting) and is less expensive to manufacture. UV lamps that can generate an ultraviolet light having a compatible wavelength are discussed in greater detail later in this specification.

In various embodiments of the invention, a fluorescent agent comprises an element, compound, or mixture that is capable of absorbing an ultraviolet light having a compatible wavelength and converting the energy absorbed from the ultraviolet light into visible light having a longer wavelength. A fluorescent agent can be of any material characterized as having the just stated capability so long as it is physiologically compatible with human skin in that the fluorescent agent is non-toxic and non-irritating when the sunscreen composition is applied to the skin.

In specific embodiments of the invention, a fluorescent agent comprises a naturally occurring fluorescent mineral. For example, fluorescent mineral can be Terlingua calcite (a calcite from Terlingua, Texas), which fluoresces in lavender when irradiated by a midwave ultraviolet light, such as an ultraviolet light having a compatible wavelength of 302 nm, and fluoresces in bright pink when irradiated by a long wave ultraviolet light, such as an ultraviolet light having a compatible wavelength of 365 nm. Examples of fluorescent minerals that fluoresce in red or orange include: axinite (calcium aluminum borate silicate); scapolite (sodium calcium aluminum silicate); kyanite (aluminum silicate); sphalerite (zinc sulphite); and petalite (lithium aluminum silicate). Examples of fluorescent minerals that fluoresce in yellow include: apatite (basic fluoro- and chloro-calcium phosphate) and cerussite (lead carbonate). Examples of fluorescent minerals that fluoresces in blue include: dumortierite (aluminum borate silicate); scheelite (calcium tungstate); smithsonite(zinc carbonate); danburite (calcium boric silicate); benitoite (barium titanium silicate); fluorite (fluorospar); and halite. Examples of fluorescent minerals that fluoresce in green to bluish green include: andalusite and chiastolite (aluminum silicate); amblygonite (basic lithium aluminum phosphorate); phenakite (beryllium silicate); variscite (hydrous aluminum phosphate); serpentine (basic magnesium silicate); amazonite (potassium aluminum silicate); amethyst (silicon dioxide); chrysoberyl (beryllium aluminum oxide); turquoise (copper-containing basic aluminum phosphate); colorless, yellow or pink tourmaline (borosilicate); amber (succinite/various resins); opal (hydrous silicon dioxide); cerussite (lead carbonate); fuchsite (potassium aluminum silicate); diopside (calcium magnesium silicate); ulexite (hydrous sodium calcium borate); aragonite (calcium carbonate); and willemite (zinc silicate).

In specific embodiments of the invention, a fluorescent agent comprises an inorganic compound doped with an inorganic fluorescence activator. A “doped” compound refers to a compound in which a “dopant,” i.e., a small amount of impurity, was intentionally introduced during production such that when irradiated by an ultraviolet light having a compatible wavelength, the compound fluoresces in visible light. When irradiated by an ultraviolet light having a compatible wavelength, an inorganic fluorescence activator emits light in the visible spectrum. An inorganic fluorescence activator, in accordance with specific embodiments of the invention, can comprise one or more of tin, thulium, niobium, cesium, cobalt, manganese, molybdenum, copper, uranium, cesium, thorium, lead, cobalt, iron, strontium, calcium, magnesium, barium, tin, yttrium, thallium, samarium, thulium, cerium, and dysprosium. An inorganic compound, in accordance with specific embodiments of the invention, can be an alkaline earth metal compound, such as an alkaline earth metal carbonate which may include at least one of calcium carbonate, barium carbonate, and magnesium carbonate. Doping an alkaline earth metal carbonate with an inorganic compound can be accomplished by wet chemistry processes known in the art such as co-precipitation, hydrothermal synthesis, and colloidal chemistry. According to some embodiments, the inorganic fluorescence activator may be contained in the crystal structure of the alkaline earth metal carbonate.

In specific embodiments of the invention, a fluorescent agent comprises precipitated calcium carbonate, an alkaline earth metal carbonate, that has been doped with manganese in an amount up to 10 mol % of the precipitated calcium carbonate. In such embodiments, the fluorescent agent fluoresces in rose to orange-red, when irradiated with an ultraviolet light having a compatible wavelength of 365 nm.

In specific embodiments of the invention, a fluorescent agent comprises a chemical fluorescent compound that is a derivative of stilbene or 4,4′-diaminostilbene; biphenyl; a 5-membered heterocycle, e.g., triazole, oxazole, or imidazole; or a 6-membered heterocycle, e.g., a coumarin, a naphthalamide, or an s-triazine. For example, the chemical fluorescent compound can be selected from the group consisting of a 4,4′-diaminostilbene-2,2′-disulfonic acid; a 2-(stilben-4-yl)naphthotriazole; a 2-(4-phenylstilben-4-yl)benzoxazole; a bis-(azol-2-yl)stilbene; a 1,4-bis(styryl)benzene; a 4,4′-bis(styryl)biphenyl; a 1,3-diphenyl-2-pyrazoline; a bis(benzooxazol-2-yl); a bis(benzimidazol-2-yl); a 2-(benzofuran-2-yl)benzimidazole; a coumarin, a carbostyril; a naphthalimide; a quaternized pyridotriazole; a pyrene compound; a 3,7-diaminodibenzothiophene-2,8-disulfonic acid-5,5-dioxide; and mixtures thereof. When irradiated with an ultraviolet light having a compatible wavelength, such as 350 nm, such a chemical fluorescent compound absorbs energy from the ultraviolet light and coverts that energy into visible light of a longer wavelength, such as at about 400 nm to about 450 nm.

In various embodiments of the invention, the sunscreen composition may further include one or more of: an emulsifier, which enables two or more constituent ingredients of the composition to be combined homogeneously, while increasing the viscosity of the composition; a film former, which keeps the composition smooth and even; an emollient, which provides a softening or soothing effect on the skin surface; a preservative, which protects the composition from microbial contamination and/or oxidation; a water-proofing agent; a pH adjuster; a perfume, and water. Suitable emulsifiers include, but are not limited to, sorbitan oleate, sorbitan sesquioleate, sorbitan isostearate, sorbitan trioleate, fatty acid soaps, e.g., potassium stearate, sodium stearate, ammonium stearate, and triethanolamine stearate; and polyol fatty acid monoesters containing fatty acid soaps, e.g., glycerol monostearate containing either potassium or sodium salt. Suitable film formers include, but are not limited to, propylene glycol, sodium lactate, acrylamide/sodium acrylate copolymer, ammonium acrylates copolymer, and cellulose gum. Suitable emollients include, but are not limited to, mineral oil, lanolin oil, coconut oil, cocoa butter, olive oil, and aloe extracts such as aloe vera. Suitable preservatives include, but are not limited to, vitamin E and its derivatives, vitamin C, butylated hydroxytoluene, and methylparaben. Suitable water-proofing agents include, but are not limited to, C30-C38 olefin/isopropyl maleate/MA copolymer.

As mentioned, the sunscreen composition, in accordance with various embodiments of the invention, can be lotion, gel, cream, or spray. The sunscreen composition, in accordance with various embodiments of the invention, may be manufactured by using processes known in the art, including emulsification process. In one aspect of the manufacturing process is that the sunscreen composition is a homogeneous composition that can be applied to skin in the form of a film. In general, the manufacturing process includes micronizing, dispersing, dissolving, mixing, heating, and/or cooling various constituent ingredients of sunscreen composition, including the sunscreen agent and the fluorescent agent. The process may comprise separate preparation phases whereby one or more subsets of the ingredients are mixed and, if necessary, heated, and thereafter the separately prepared subsets are mixed together to form the sunscreen composition. For example, the separate phases may include one or more oil phases containing lipophilic ingredients and one or more aqueous phase containing hydrophilic ingredients. The sunscreen composition, in accordance with various embodiments of the invention, is packaged in a suitable container, such as a bottle or tube, which is discussed below.

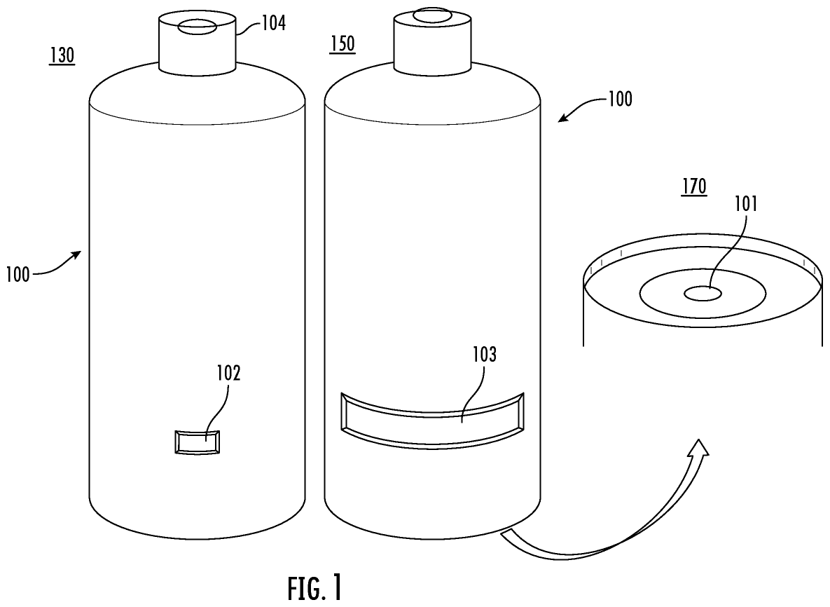

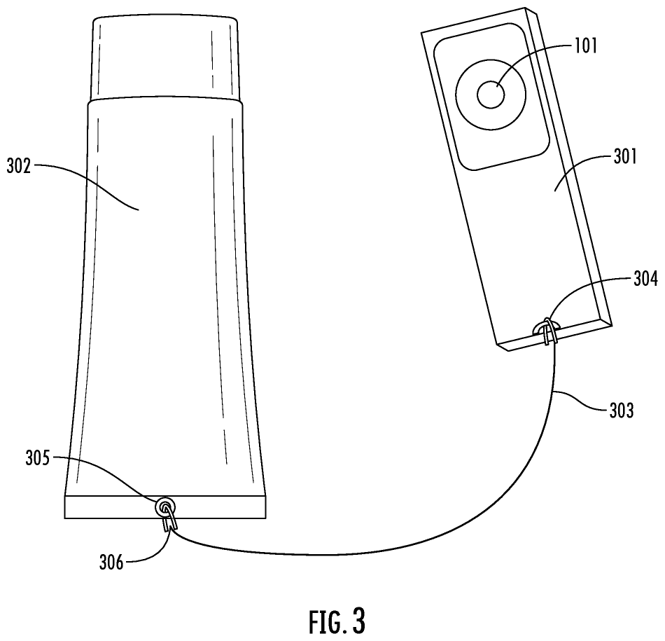

FIG. 1 illustrates an example of container for packaging and containing sunscreen composition, in accordance with specific embodiments disclosed herein. The embodiment shown in FIG. 3 includes a UV lamp that integrated into the container. View 130 illustrates one side of the container 100, view 150 illustrates a side of the container opposite to the side of the container shown in view 130, and view 170 illustrates the bottom side of the container. The container includes a light-emitting diode (LED) 101, a power switch 102 that can be used to turn on or off power to the LED, and an access door 103 to slidably insert or remove a tray containing batteries. The door forms a wall of the tray. The lid of the container's cap 104 can be pushed to open to dispense sunscreen composition contained therein and thereafter, can be pushed to close. In some embodiments, the cap can, instead, include a spray nozzle or a manually operable pump. The container 100 can be refiled when the sunscreen composition runs low or the container becomes empty by unscrewing the cap 104 from the container. The container can be made from plastics such as low-density polyethylene (LDPE), medium-density polyethylene (MDPE), high-density polyethylene (HDPE), and ultra-high molecular weight polyethylene (UHMW), using process known in the art such as injection molding processes, blown molding processes, etc.

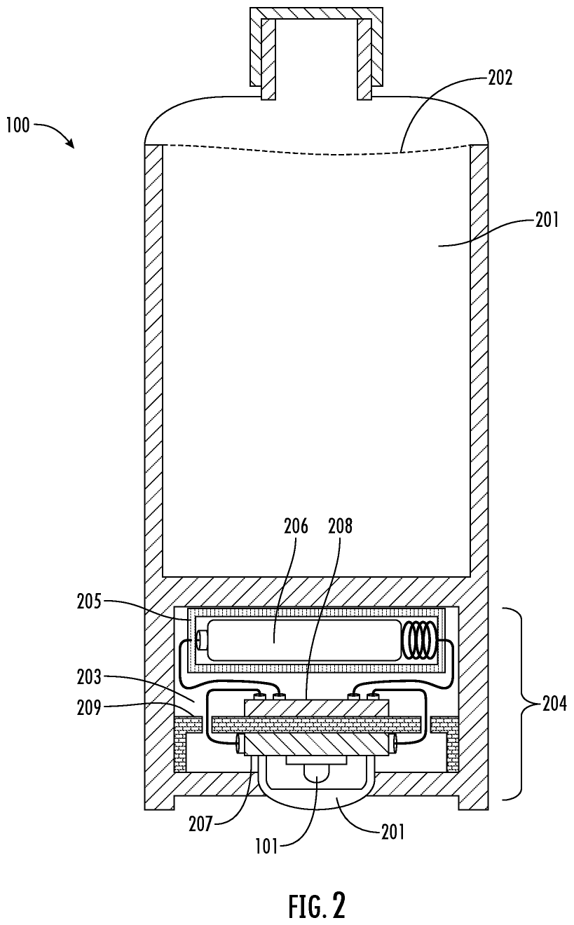

FIG. 2 illustrates an example cross-sectional view of the container in FIG. 1. The container has an upper chamber 201 for containing sunscreen composition 202 and a lower chamber 203 that houses various components of UV lamp 204. The UV lamp comprises a LED 101, a battery tray 205, one or more batteries 206, a printed circuit board 207 that houses the LED, a circuit board 208, and a heat sink 209 for dissipating heat generated by the LED. Circuit board 208 includes various circuitry necessary for the operation of the LED including, for example, current regulator and circuitry to boost power supply from the battery to the LED. In some embodiments, the battery 206 can be rechargeable, and in such embodiments, the circuit board 208 can include charging circuitry.

As discussed above, fluorescent agents, in various embodiments of the invention, are selected such that they fluoresce visible light when they are irradiated with an ultraviolet light having a compatible wavelength between around 280 nm and around 400 nm (which comprises the longwave and midwave regions the ultraviolet spectrum). Generating ultraviolet light within this range of wavelength reduces the cost of ultraviolet light source in that a relatively inexpensive LED bulb can be used. LED 101 can be chosen such that its peak wavelength matches a compatible wavelength at which the fluorescent agent included in the sunscreen composition contained in the container 100 fluoresces, and consequently, the LED, when powered on, emits an ultraviolet light having the compatible wavelength. In specific embodiment of the invention, the peak wavelength of the LED is 365±5 nm. In specific embodiment of the invention, the peak wavelength of the LED is 308±5 nm. In specific embodiments, the UV lamp can include multiple LEDs, each having a different peak wavelength. The UV lamp includes a lens 201 collimates the radiation from the LED 101 into a sharply focused beam. In specific embodiments, the lens has a viewing angle of 30°.

FIG. 3 illustrates an example of a container for packaging and containing sunscreen composition and a detachable UV lamp, in accordance with specific embodiments disclosed herein. In the embodiment depicted in FIG. 3, a UV lamp 301 is attached to the container 302 with a rope 303, which may be made of plastic or metal. On one end, the rope 303 is permanently attached to the UV lamp 301 by a ring 304, and on the other end, the rope is attached to a hole 305 of the container 301 by a hook 306. The hook 306 can be opened and closed such that the rope 303, and therefore, UV lamp 301, can be detached from the container 302 and thereafter, can be reattach the lamp to a new container. In this way, a container can be discarded when it runs low on, or out of, sunscreen composition, but the UV lamp, which can be more expensive than the sunscreen composition, can be reused with a new container filled with sunscreen composition.

In specific embodiments of the invention, the container can include an indicator for identifying whether a recommended amount of sunscreen has been applied. For example, the packaging for the container or the container itself could include an indicator in the form of an illustration with different shades and/or intensities of color. The different shades or intensities of color could be associated with different levels of the composition in the skin with insufficient levels of composition being associated with low intensities or lighter shades, and sufficient levels being associated with stronger intensities or darker shades. A user could then utilize the indicator to compare against the skin after the composition had been applied to confirm whether or not a sufficient amount of the composition had been applied to the skin.

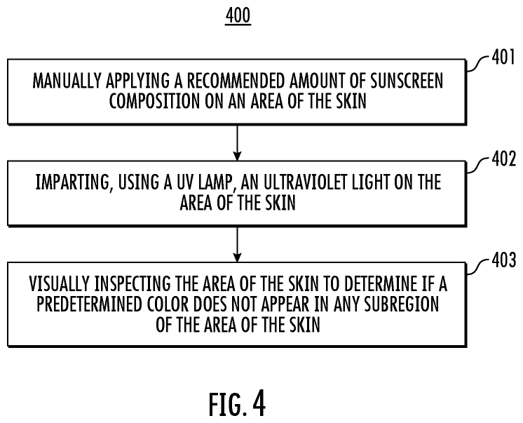

FIG. 4 illustrates a flowchart 400 for a method for determining adequacy of an application of a sunscreen composition on skin, in accordance with specific embodiments disclosed herein. The method of flowchart 400 starts at step 401 of manually applying a recommended amount of the sunscreen composition to an area of the skin. “Manually” means by hand or other manual means. For example, the sunscreen composition could be dispensed in aerosolized form such that the manual application involved spraying the sun tan lotion onto an area of skin. A “recommended amount” of the sunscreen composition applied on skin can provide adequate protection from the harmful effects of the sun's ultraviolet light, such as UVA and UVB rays. For example, a recommended amount can be 2 mg/cm² of the sunscreen composition. The recommended amount can vary depending upon the specific formulation of the sunscreen composition, including the amount of sunscreen agent included in the composition. The recommended amount can be the amount recommended by the CDC and FDA for a given person and situation.

Flowchart 400 continues to step 402 of imparting, using a UV lamp, an ultraviolet light on the area of the skin. During this step, a UV lamp, such as an UV lamp 204, 301, is used to impart an ultraviolet light of a compatible wavelength over the area of the skin where the sunscreen composition has been applied in step 401. The UV lamp is powered on so that its LED is activated so that it emits ultraviolet light of the compatible wavelength, and the lamp is held a short distance, such as 1-4 inches, away from the area of the skin so that the emitted ultraviolet light imparts on the area.

Flowchart 400 continues to step 403 of visually inspecting, during the imparting of the ultraviolet light, the area of the skin to determine if the predetermined color does not appear in any subregion of the area of the skin. “Visual inspection” means by an inspection by the naked eye. During this step, it is determined whether or not the sunscreen composition has been applied, in step 401, adequately across the entirety of the area of the skin, that is, whether all of the area received adequate coverage of the composition. This determination is based on the color of the visible light emitted by the specific fluorescent agent of the sunscreen composition applied on the skin. For example, if the fluorescent agent is Terlingua calcite, the agent, as discussed above, fluoresces in bright pink when irradiated by an ultraviolet light having a compatible wavelength of 365 nm, and the UV lamp imparts an ultraviolet light having 365 nm wavelength in step 402. In this example, bright pink is the predetermined color. Accordingly, during the visual inspection, the user of the sunscreen composition looks to see if any spot or subregion of the area of the skin does not appear in bright pink color. If the inspection reveals that there is such a spot, the sunscreen composition has not been applied adequately. If, on the other hand, the inspection reveals that there is not such a spot (i.e., the entirety of the area of the skin appears in bright pink color approximately uniformly), then the sunscreen composition has been applied adequately. Flowchart ends at step 403. If the area of the skin was not adequately covered by the sunscreen composition, steps 401-403 can be repeated by applying more composition to the subregion(s) of the area of the skin does not appear in bright pink color. If, however, the area of the skin was adequately covered, the user may perform steps 401-403 in another area of the skin.

While the specification has been described in detail with respect to specific embodiments of the invention, it will be appreciated that those skilled in the art, upon attaining an understanding of the foregoing, may readily conceive of alterations to, variations of, and equivalents to these embodiments. These and other modifications and variations to the present invention may be practiced by those skilled in the art, without departing from the scope of the present invention, which is more particularly set forth in the appended claims.

What is claimed is:

1. A sunscreen composition for application to human skin, comprising:
a sunscreen agent selected from the group consisting of UVA sunscreen agent, UVB sunscreen agent, UVA-UVB sunscreen agent, and mixture thereof; and
a fluorescent agent in an effective amount;
wherein the fluorescent agent emits a visible light when irradiated with an ultraviolet light having a compatible wavelength.

2. The sunscreen composition of claim 1,
wherein the effective amount is from 0.01 wt. % to 30 wt. % of the sunscreen composition.

3. The sunscreen composition of claim 1,
wherein the compatible wavelength is from 280 nm to 400 nm.

4. The sunscreen composition of claim 1,
wherein the UVA sunscreen agent is selected from the group consisting of Benzophenone-1, Benzophenone-2, Benzophenone-3, Benzophenone-4, Benzophenone-6, Benzophenone-8, Butyl methyl dibenzoyl methane, Disodium phenyl dibenzimidazole tetrasulfonate, Menthyl anthranilate, 4-isopropyl dibenzoyl methane, and Terephthalylidene dicamphor sulfonic acid;
wherein the UVB sunscreen agent is selected from the group consisting of DEA methoxyinnamate, Ethyl dihydroxypropl PABA, Glycerol PABA, Homosalate, Octocrylene, Octyl dimethyl PABA, OCtyl methoxycinnamate, Octyl salicylate, PABA, 2-Phenyl-benzimidazole-5-Sulphonic acid, TEA Salicylate, 3-(4-methylbenzlidene)camphor or 3-(4-methylbenzylidene)boran-2-one, and Etocrylene; and
wherein the UVA-UVB sunscreen agent is selected from the group consisting of Benzophenone-3, Benzophenone-4, Benzophenone-8, 3-Benzylidene camphor, Bis-ethylhexyloxyphenol methoxyphenyl triazine, Cinoxate, Drometrizole trisiloxane, Methylene bis-benzotriazolyl, Tetramethylbutylphenol, Octocrylene, and Phenylbenzimidazole sulfonic acid.

5. The sunscreen composition of claim 1,
wherein the fluorescent agent comprises a naturally occurring fluorescent mineral.

6. The sunscreen composition of claim 5,
wherein the naturally occurring fluorescent mineral is Terlingua calcite.

7. The sunscreen composition of claim 1,
wherein the fluorescent agent comprises an inorganic compound doped with an inorganic fluorescence activator.

8. The sunscreen composition of claim 7,
wherein the inorganic compound is precipitated calcium carbonate;
wherein the inorganic fluorescence activator is manganese; and
wherein the inorganic fluorescence activator comprises up to 10 mol % of the inorganic compound.

9. The sunscreen composition of claim 1,
wherein the fluorescent agent comprises a chemical fluorescent compound selected from the group consisting of 4,4′-diaminostilbene-2,2′-disulfonic acid; 2-(stilben-4-yl)naphthotriazole; 2-(4-phenylstilben-4-yl)benzoxazole; bis-(azol-2-yl)stilbene; 1,4-bis(styryl)benzene; 4,4′-bis(styryl)biphenyl; a 1,3-diphenyl-2-pyrazoline; bis(benzooxazol-2-yl); bis(benzimidazol-2-yl); 2-(benzofuran-2-yl)benzimidazole; coumarin, carbostyril; naphthalimide; quaternized pyridotriazole; pyrene compound; 3,7-diaminodibenzothiophene-2,8-disulfonic acid-5,5-dioxide; and mixtures thereof.

10. The sunscreen composition of claim 1,
wherein the sunscreen composition is in one of a lotion, a gel, a cream, and a spray.

11. The sunscreen composition of claim 1 further comprising one or more of emulsifier, film former, emollient, preservative, water-proofing agent, pH adjuster, perfume, and water.

12. A method for determining adequacy of an application on skin of a sunscreen composition comprising (i) a sunscreen agent selected from the group consisting of UVA sunscreen agent, UVB sunscreen agent, UVA-UVB sunscreen agent, and mixture thereof, and (ii) a fluorescent agent in an effective amount, wherein the fluorescent agent emits a visible light of a predetermined color, when irradiated with an ultraviolet light having a compatible wavelength, the method comprising:
manually applying a recommended amount of the sunscreen composition on an area of the skin;
imparting, using a UV lamp, the ultraviolet light on the area of the skin; and
visually inspecting, during the imparting the ultraviolet light, the area of the skin to determine if the predetermined color does not appear in any subregion of the area of the skin.

13. The method of claim 12,
wherein the effective amount is from 0.01 wt. % to 30 wt. % of the sunscreen composition.

14. The method of claim 12,
wherein the compatible wavelength is from 280 nm to 400 nm.

15. The method of claim 12,
wherein the UVA sunscreen agent is selected from the group consisting of Benzophenone-1, Benzophenone-2, Benzophenone-3, Benzophenone-4, Benzophenone-6, Benzophenone-8, Butyl methyl dibenzoyl methane, Disodium phenyl dibenzimidazole tetrasulfonate, Menthyl anthranilate, 4-isopropyl dibenzoyl methane, and Terephthalylidene dicamphor sulfonic acid;
wherein the UVB sunscreen agent is selected from the group consisting of DEA methoxyinnamate, Ethyl dihydroxypropl PABA, Glycerol PABA, Homosalate, Octocrylene, Octyl dimethyl PABA, OCtyl methoxycinnamate, Octyl salicylate, PABA, 2-Phenyl-benzimidazole-5-Sulphonic acid, TEA Salicylate, 3-(4-methylbenzlidene)camphor or 3-(4-methylbenzylidene)boran-2-one, and Etocrylene; and
wherein the UVA-UVB sunscreen agent is selected from the group consisting of Benzophenone-3, Benzophenone-4, Benzophenone-8, 3-Benzylidene camphor, Bis-ethylhexyloxyphenol methoxyphenyl triazine, Cinoxate, Drometrizole trisiloxane, Methylene bis-benzotriazolyl, Tetramethylbutylphenol, Octocrylene, and Phenylbenzimidazole sulfonic acid.

16. The method of claim 12,
wherein the fluorescent agent comprises a naturally occurring fluorescent mineral.

17. The method of claim 16,
wherein the naturally occurring fluorescent mineral is Terlingua calcite.

18. The method of claim 12,
wherein the fluorescent agent comprises an inorganic compound doped with an inorganic fluorescence activator.

19. The method of claim 18,
wherein the inorganic compound is precipitated calcium carbonate;
wherein the inorganic fluorescence activator is manganese; and
wherein the inorganic fluorescence activator comprises up to 10 mol % of the inorganic compound.

20. The method of claim 12,
wherein the fluorescent agent comprises a chemical fluorescent compound selected from the group consisting of 4,4′-diaminostilbene-2,2′-disulfonic acid; 2-(stilben-4-yl)naphthotriazole; 2-(4-phenylstilben-4-yl)benzoxazole; bis-(azol-2-yl)stilbene; 1,4-bis(styryl)benzene; 4,4′-bis(styryl)biphenyl; a 1,3-diphenyl-2-pyrazoline; bis(benzooxazol-2-yl); bis(benzimidazol-2-yl); 2-(benzofuran-2-yl)benzimidazole; coumarin, carbostyril; naphthalimide; quaternized pyridotriazole; pyrene compound; 3,7-diaminodibenzothiophene-2,8-disulfonic acid-5,5-dioxide; and mixtures thereof.

21. The method of claim 12,
wherein the sunscreen composition is in one of a lotion, a gel, a cream, and a spray.

22. The method of claim 12,
wherein the sunscreen composition further comprises one or more of emulsifier, film former, emollient, preservative, water-proofing agent, pH adjuster, perfume, and water.

23. A portable device comprising:
a container containing a sunscreen composition, the sunscreen composition comprising (i) a sunscreen agent selected from the group consisting of UVA sunscreen agent, UVB sunscreen agent, UVA-UVB sunscreen agent, and mixture thereof, and (ii) a fluorescent agent; and
a UV lamp attached to the container; and
wherein the UV lamp, when powered on, emits an ultraviolet light having a compatible wavelength;
wherein the sunscreen composition emits a visible light when irradiated with the ultraviolet light emitted by the UV lamp.

24. The portable device of claim 23,
wherein the UV lamp is integrated into the container.

25. The portable device of claim 23,
wherein the UV lamp is detachable from the container.

26. The portable device of claim 23,
wherein the UV lamp comprises an LED; and
wherein the LED has a peak wavelength that matches the compatible wavelength.

27. The portable device of claim 23,
wherein the compatible wavelength is from 280 nm to 400 nm.

28. The portable device of claim 23,
wherein the fluorescent agent comprises a naturally occurring fluorescent mineral.

29. The portable device of claim 23,
wherein the fluorescent agent comprises an inorganic compound doped with an inorganic fluorescence activator.

30. The portable device of claim 23,
wherein the fluorescent agent comprises a chemical fluorescent compound selected from the group consisting of 4,4′-diaminostilbene-2,2′-disulfonic acid; 2-(stilben-4-yl)naphthotriazole; 2-(4-phenylstilben-4-yl)benzoxazole; bis-(azol-2-yl)stilbene; 1,4-bis(styryl)benzene; 4,4′-bis(styryl)biphenyl; a 1,3-diphenyl-2-pyrazoline; bis(benzooxazol-2-yl); bis(benzimidazol-2-yl); 2-(benzofuran-2-yl)benzimidazole; coumarin, carbostyril; naphthalimide; quaternized pyridotriazole; pyrene compound; 3,7-diaminodibenzothiophene-2,8-disulfonic acid-5,5-dioxide; and mixtures thereof.
